(19) United States
(12) Patent Application Publication
(10) Pub. No.: US 2025/0014213 A1
(43) Pub. Date: Jan. 9, 2025

(54) IMAGE PROCESSING APPARATUS, IMAGE PROCESSING METHOD, AND NON-TRANSITORY STORAGE MEDIUM

(71) Applicant: NEC Corporation, Minato-ku, Tokyo (JP)

(72) Inventors: Ryo KAWAI, Tokyo (JP); Noboru YOSHIDA, Tokyo (JP); Jianquan LIU, Tokyo (JP)

(73) Assignee: NEC Corporation, Minato-ku, Tokyo (JP)

(21) Appl. No.: 18/709,881

(22) PCT Filed: Mar. 7, 2022
(86) PCT No.: PCT/JP2022/009739

§ 371 Date: May 14, 2024

(51) Int. Cl. G06T 7/73; G06F 3/14

(52) U.S. Cl. CPC G06T 7/73; G06F 3/14; G06T 2207/20044; G06T 2207/30196

(57) ABSTRACT

The present invention provides an image processing apparatus (10) including: a screen generation unit (11) that generates a screen including a playback region playing back and displaying a moving image including a plurality of frame images and a missing key point display region indicating a key point of a human body not being detected in a human body included in the frame image displayed in the playback region, and causes a display unit to display the generated screen; and an input reception unit (12) that receives an input specifying a section to be extracted from the moving image.

### TECHNICAL FIELD

The present invention relates to an image processing apparatus, an image processing method, and a storage medium.

### BACKGROUND ART

Techniques related to the present invention are disclosed in Patent Document 1 and Non-Patent Document 1.

In Patent Document 1, a technique of computing a feature value of each of a plurality of key points of a human body included in an image, searching for an image including a human body having a similar pose or a human body having a similar movement, based on the computed feature value, and collectively classifying the human bodies with a similar pose or movement. Non-Patent Document 1 discloses a technique related to skeleton estimation of a person.

### RELATED DOCUMENT

### Patent Document

Patent Document 1: International Patent Publication No. WO2021/084677

### Non-Patent Document

Non-Patent Document 1: Zhe Cao, Tomas Simon, Shih-En Wei, Yaser Sheikh, “Realtime Multi-Person 2D Pose Estimation using Part Affinity Fields”, The IEEE Conference on Computer Vision and Pattern Recognition (CVPR), 2017, pp. 7291 to 7299

### DISCLOSURE OF THE INVENTION

### Technical Problem

According to the technique disclosed in Patent Document 1 described above, by registering an image including a human body having a desired pose or a desired movement as a template image in advance, it is possible to detect a human body having the desired pose or the desired movement from an image to be processed. As a result of studying the technique disclosed in Patent Document 1, the present inventor has newly found that accuracy of detection is deteriorated unless an image having certain quality is registered as a template image, and there is room for improvement in workability of work for preparing such a template image.

Both of Patent Document 1 and Non-Patent Document 1 described above do not disclose a problem related to a template image and a solution thereof, and therefore, there is a problem that the problem described above cannot be solved.

In view of the problem described above, an example object of the present invention is to provide an image processing apparatus, an image processing method, and a storage medium that solve a problem of workability of work for preparing a template image having certain quality.

### Solution to Problem

According to one aspect of the present invention, there is provided an image processing apparatus including:
a screen generation unit that generates a screen including a playback region playing back and displaying a moving image including a plurality of frame images, and a missing key point display region indicating a key point of a human body not being detected in a human body included in the frame image displayed in the playback region, and causes a display unit to display the generated screen; and
an input reception unit that receives an input specifying a section to be extracted from the moving image.

Further, according to one aspect of the present invention, there is provided an image processing method including,
by a computer:
   generating a screen including a playback region playing back and displaying a moving image including a plurality of frame images, and a missing key point display region indicating a key point of a human body not being detected in a human body included in the frame image displayed in the playback region, and causing a display unit to display the generated screen; and
   receiving an input specifying a section to be extracted from the moving image.

Further, according to one aspect of the present invention, there is provided a storage medium storing a program causing a computer to function as:
a screen generation unit that generates a screen including a playback region playing back and displaying a moving image including a plurality of frame images, and a missing key point display region indicating a key point of a human body not being detected in a human body included in the frame image displayed in the playback region, and causes a display unit to display the generated screen; and
an input reception unit that receives an input specifying a section to be extracted from the moving image.

### Advantageous Effects of Invention

According to one aspect of the present invention, an image processing apparatus, an image processing method, and a storage medium that solve a problem of workability of work for preparing a template image having certain quality are acquired.

### BRIEF DESCRIPTION OF THE DRAWINGS

The above-described object and another object, a feature, and an advantage will become more apparent from the following description of public example embodiments and the accompanying drawings thereof.

FIG. 1 It is a diagram illustrating one example of a functional block diagram of an image processing apparatus.

FIG. 2 It is one example of a UI screen generated by the image processing apparatus.

FIG. 3 It is a diagram illustrating one example of a hardware configuration of the image processing apparatus.

FIG. 4 It is a diagram illustrating another example of a functional block diagram of the image processing apparatus.

FIG. 5 It is a diagram illustrating one example of a skeleton structure of a human body model detected by the image processing apparatus.

FIG. 6 It is a diagram illustrating one example of a skeleton structure of a human body model detected by the image processing apparatus.

FIG. 7 It is a diagram illustrating one example of a skeleton structure of a human body model detected by the image processing apparatus.

FIG. 8 It is a diagram illustrating one example of a skeleton structure of a human body model detected by the image processing apparatus.

FIG. 9 It is a diagram schematically illustrating one example of information processed by the image processing apparatus.

FIG. 10 It is a flowchart illustrating one example of a flow of processing of the image processing apparatus.

FIG. 11 It is another example of a UI screen generated by the image processing apparatus.

FIG. 12 It is another example of a UI screen generated by the image processing apparatus.

FIG. 13 It is another example of a UI screen generated by the image processing apparatus.

FIG. 14 It is another example of a UI screen generated by the image processing apparatus.

FIG. 15 It is another example of a UI screen generated by the image processing apparatus.

FIG. 16 It is another example of a UI screen generated by the image processing apparatus.

FIG. 17 It is another example of a UI screen generated by the image processing apparatus.

FIG. 18 It is another example of a UI screen generated by the image processing apparatus.

FIG. 19 It is a diagram schematically illustrating one example of moving object state information processed by the image processing apparatus.

FIG. 20 It is another example of a UI screen generated by the image processing apparatus.

### DESCRIPTION OF EMBODIMENTS

Hereinafter, example embodiments of the present invention will be described with reference to the drawings. Note that, in all the drawings, a similar component is denoted by a similar reference sign, and description thereof will be omitted as appropriate.

### First Example Embodiment

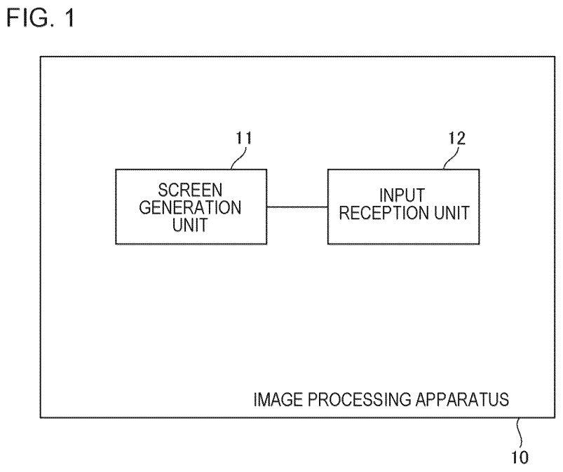

FIG. 1 is a functional block diagram illustrating an overview of an image processing apparatus 10 according to a first example embodiment. As illustrated in FIG. 1, the image processing apparatus 10 includes a screen generation unit 11, and an input reception unit 12. The screen generation unit 11 generates a screen including a playback region displaying a moving image including a plurality of frame images, and a missing key point display region indicating a key point of a human body not being detected in the human body included in the frame image displayed in the playback region, and causes a display unit to display the generated screen. The input reception unit 12 receives an input specifying a section to be extracted from the moving image.

According to the image processing apparatus 10, it is possible to solve a problem of workability of work for preparing a template image having certain quality.

### Second Example Embodiment

### “Overview”

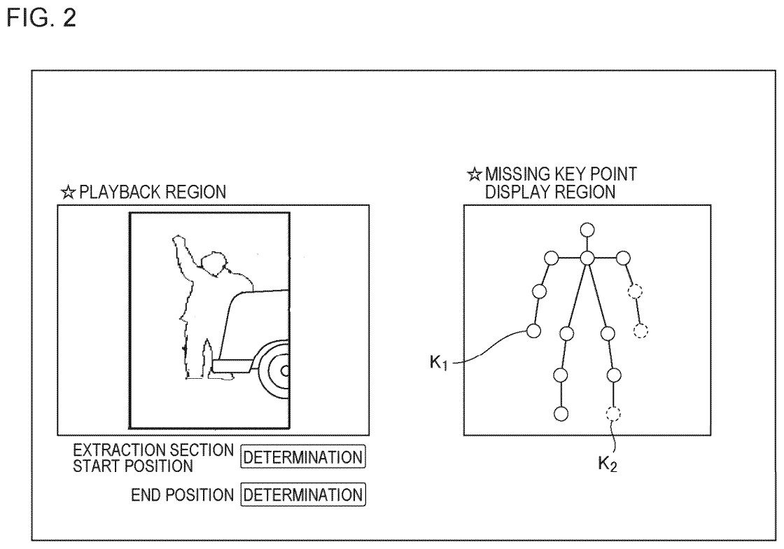

As illustrated in FIG. 2, for example, an image processing apparatus 10 generates a user interface (UI) screen including a playback region playing back and displaying a moving image, and a missing key point display region indicating a key point of a human body not being detected in the human body included in the frame image displayed in the playback region, and causes a display unit to display the generated screen. Then, the image processing apparatus 10 can receive an input specifying a section to be extracted as a template image from the moving image via such a UI screen.

A user can determine a portion in a moving image including a human body having a desired pose or a desired movement and having a good detection state of a key point while referring to the playback region and the missing key point display region, and extract the determined portion as a template image.

### “Hardware Configuration”

Next, one example of a hardware configuration of the image processing apparatus 10 will be described. Each functional unit of the image processing apparatus 10 is achieved by any combination of hardware and software, mainly including a central processing unit (CPU) of any computer, a memory, a program loaded into a memory, a storage unit, such as a hard disk, storing the program (in addition to a program stored from a stage of shipping an apparatus in advance, a program downloaded from a storage medium such as a compact disc (CD) or a server on the Internet can also be stored), and an interface for network connection. Then, it is understood by a person skilled in the art that there are various modification examples to an implementation method and apparatus.

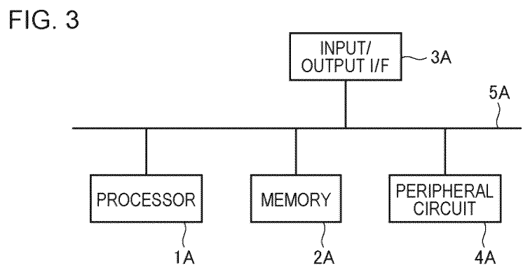

FIG. 3 is a block diagram illustrating a hardware configuration of the image processing apparatus 10. As illustrated in FIG. 3, the image processing apparatus 10 includes a processor 1A, a memory 2A, an input/output interface 3A, a peripheral circuit 4A, and a bus 5A. The peripheral circuit 4A includes various modules. The image processing apparatus 10 may not include the peripheral circuit 4A. Note that, the image processing apparatus 10 may be configured by a plurality of apparatuses that are physically and/or logically separated. In this case, each of the plurality of apparatuses can include the hardware configuration described above.

The bus 5A is a data transmission path through which the processor 1A, the memory 2A, the peripheral circuit 4A, and the input/output interface 3A transmit and receive data to and from one another. The processor 1A is, for example, an arithmetic processing apparatus such as a CPU or a graphics processing unit (GPU). The memory 2A is, for example, a memory such as a random access memory (RAM) or a read only memory (ROM). The input/output interface 3A includes an interface for acquiring information from an input apparatus, an external apparatus, an external server, an external sensor, a camera, and the like, an interface for outputting information to an output apparatus, an external apparatus, an external server, and the like, and the like. The input apparatus is, for example, a keyboard, a mouse, a microphone, a physical button, a touch panel, and the like. The output apparatus is, for example, a display, a speaker, a printer, a mailer, and the like. The processor 1A can issue a command to each module, and perform an arithmetic operation, based on an arithmetic operation result thereof.

### “Functional Configuration”

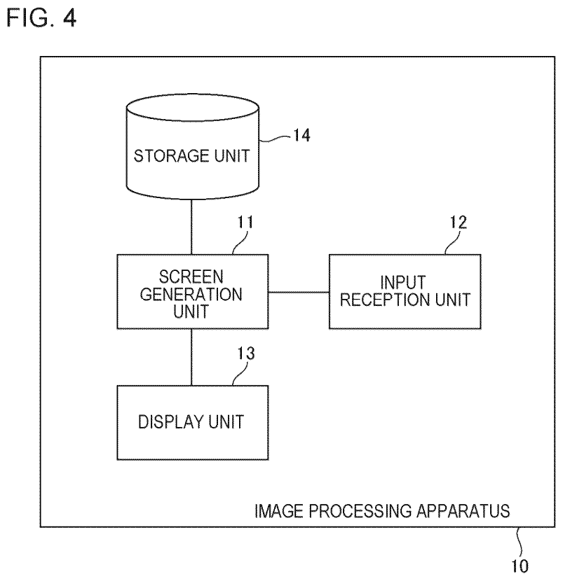

FIG. 4 is a functional block diagram illustrating an overview of the image processing apparatus 10 according to the second example embodiment. As illustrated in FIG. 4, the image processing apparatus 10 includes a screen generation unit 11, an input reception unit 12, a display unit 13, and a storage unit 14. Note that, the image processing apparatus 10 may not include the storage unit 14. In this case, an external apparatus configured to be communicable with the image processing apparatus 10 includes the storage unit 14. Further, the image processing apparatus 10 may not include the display unit 13. In this case, an external apparatus configured to be communicable with the image processing apparatus 10 includes the display unit 13.

The storage unit 14 stores a result of detection processing of a key point of a human body performed on each of a plurality of frame images included in a moving image.

A “moving image” is an original image of a template image. The template image is an image (a concept including a still image and a moving image) registered in advance in the technique disclosed in Patent Document 1 described above, and an image including a human body having a desired pose or a desired movement (a pose or a movement desired to be detected by a user).

A skeleton structure detection unit performs the detection processing of a key point of a human body. The image processing apparatus 10 may include the skeleton structure detection unit, or another apparatus physically and/or logically separated from the image processing apparatus 10 may include the skeleton structure detection unit.

The skeleton structure detection unit detects, for each frame image, N (N is an integer of 2 or more) key points of a human body included in each frame image. The processing by the skeleton structure detection unit is achieved by using the technique disclosed in Patent Document 1. Although details are omitted, in the technique disclosed in Patent Document 1, detection of a skeleton structure is performed by using a skeleton estimation technique such as OpenPose disclosed in Non-Patent Document 1. The skeleton structure detected by the technique consists of a “key point” being a characteristic point such as a joint, and a “bone (bone link)” indicating a link between the key points.

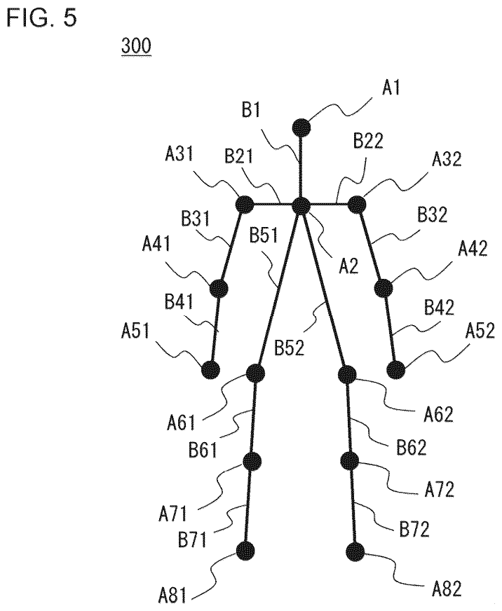

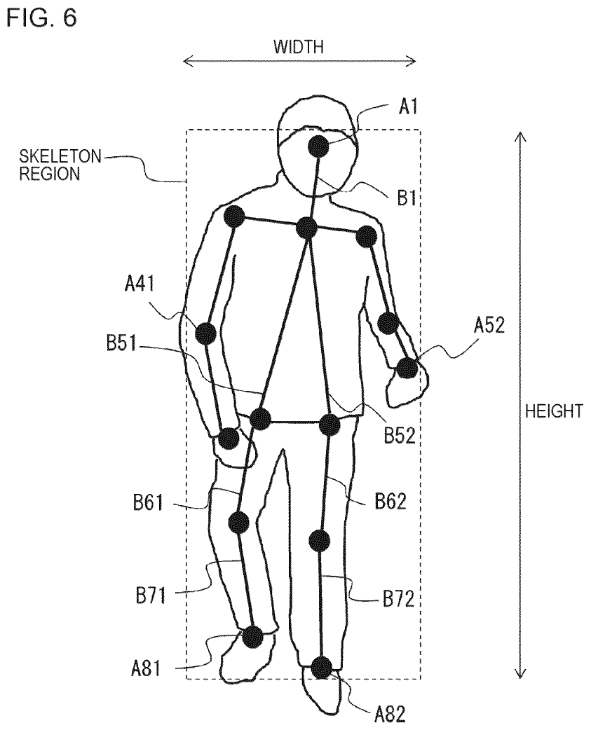

FIG. 5 illustrates a skeleton structure of a human body model 300 detected by the skeleton structure detection unit, and FIGS. 6 to 8 illustrate examples of detection of the skeleton structure. The skeleton structure detection unit detects, by using a skeleton estimation technique such as OpenPose, the skeleton structure of the human body model (two-dimensional skeleton model) 300 as illustrated in FIG. 5 from a two-dimensional image. The human body model 300 is a two-dimensional model consisted of a key point such as a joint of a person and a bone connecting each key point.

For example, the skeleton structure detection unit extracts a keypoint that may be a key point from an image, and detects N key points of a human body by referring to information acquired by performing machine learning on the image of the key point. The N key points to be detected are predetermined. The number of key points to be detected (i.e., the number of N) and which part of the human body is a key point to be detected varies, and any variation can be adopted.

Hereinafter, as illustrated in FIG. 5, it is assumed that a head A1, a neck A2, a right shoulder A31, a left shoulder A32, a right elbow A41, a left elbow A42, a right hand A51, a left hand A52, a right waist A61, a left waist A62, a right knee A71, a left knee A72, a right foot A81, and a left foot A82 are defined as N key points (N=14) to be detected. Note that, in the human body model 300 illustrated in FIG. 5, a bone B1 connecting the head A1 and the neck A2, a bone B21 connecting the neck A2 and the right shoulder A31, a bone B22 connecting the neck A2 and the left shoulder A32, a bone B31 connecting the right shoulder A31 and the right elbow A41, a bone B32 connecting the left shoulder A32 and the left elbow A42, a bone B41 connecting the right elbow A41 and the right hand A51, a bone B42 connecting the left elbow A42 and the left hand A52, a bone B51 connecting the neck A2 and the right waist A61, a bone B52 connecting the neck A2 and the left waist A62, a bone B61 connecting the right waist A61 and the right knee A71, a bone B62 connecting the left waist A62 and the left knee A72, a bone B71 connecting the right knee A71 and the right foot A81, and a bone B72 connecting the left knee A72 and the left foot A82 are further defined as bones of a person acquired by connecting the key points.

FIG. 6 is an example of detecting a person in a standing-up state. In FIG. 6, an image of a person standing-up is captured from a front, each of the bone B1, the bone B51 and the bone B52, the bone B61 and the bone B62, and the bone B71 and the bone B72 viewed from the front is detected without overlapping with each other, and the bone B61 and the bone B71 of the right foot slightly bend more than the bone B62 and the bone B72 of the left foot.

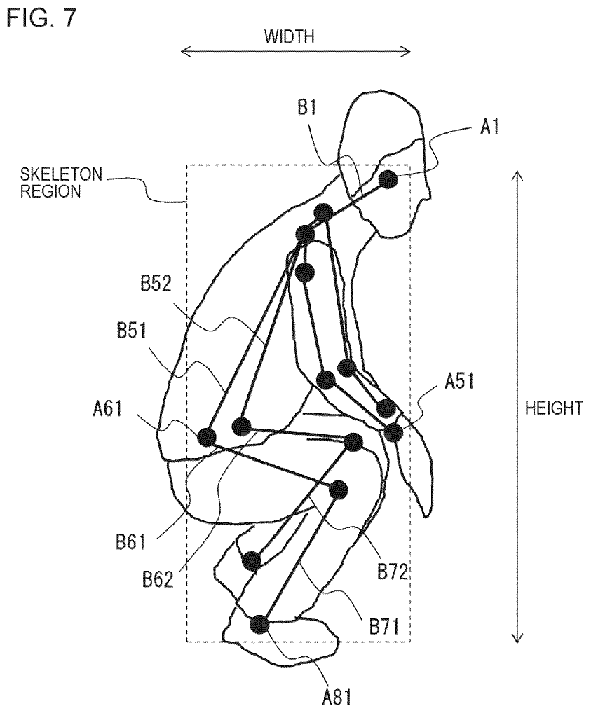

FIG. 7 is an example of detecting a person in a squatting-down state. In FIG. 7, an image of a person squatting-down is captured from a right side, each of the bone B1, the bone B51 and the bone B52, the bone B61 and the bone B62, and the bone B71 and the bone B72 viewed from the right side is detected, and the bone B61 and the bone B71 of the right foot and the bone B62 and the bone B72 of the left foot greatly bend and overlap with each other.

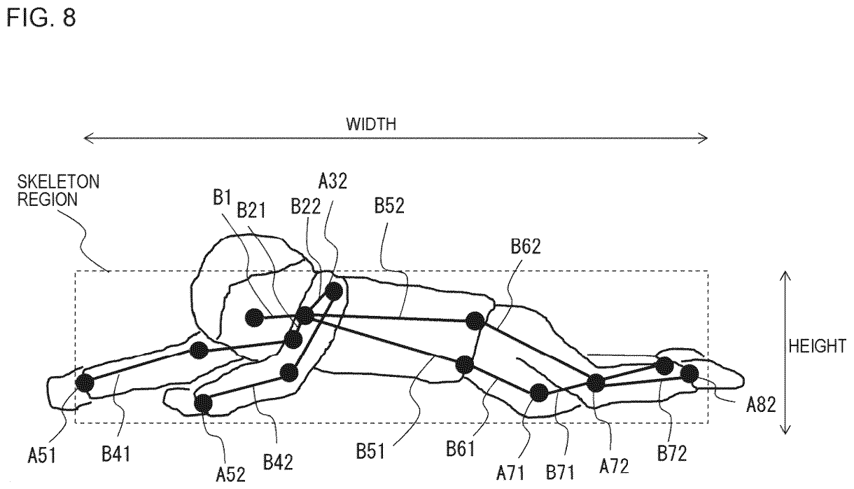

FIG. 8 is an example of detecting a person in a sleeping state. In FIG. 8, an image of a sleeping person is captured from a left obliquely front, each of the bone B1, the bone B51 and the bone B52, the bone B61 and the bone B62, and the bone B71 and the bone B72 viewed from the left obliquely front is detected, and the bone B61 and the bone B71 of the right foot and the bone B62 and the bone B72 of the left foot bend and overlap with each other.

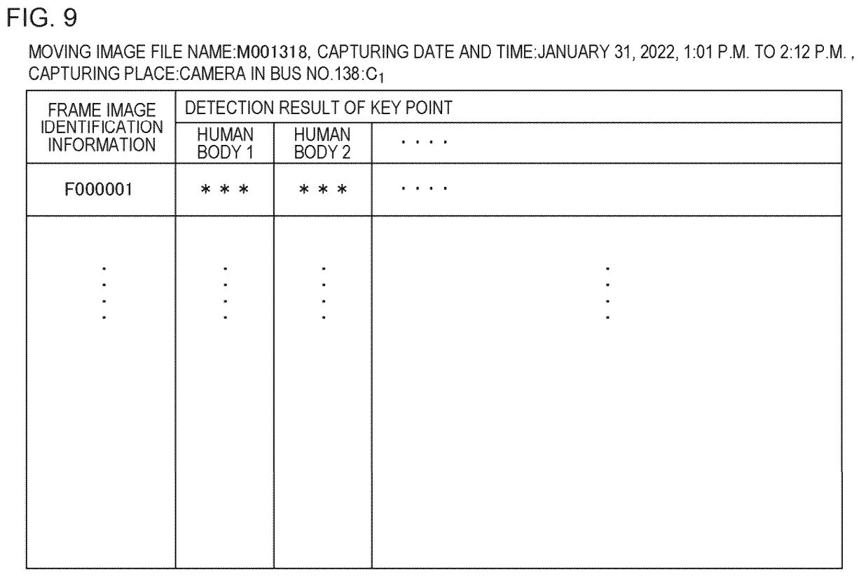

FIG. 9 schematically illustrates one example of information stored in the storage unit 14. As illustrated in FIG. 9, the storage unit 14 stores a detection result of a key point of a human body for each frame image (for each piece of frame image identification information). When a plurality of human bodies are included in one frame image, detection results of a key point of each of the plurality of human bodies are stored in association with the frame image.

The storage unit 14 stores, as a detection result of a key point of a human body, data capable of reproducing the human body model 300 having a predetermined pose as illustrated in FIGS. 6 to 8. In the detection result of a key point of a human body, which key point among the N key points to be detected is detected and which key point is not detected is indicated. Further, the storage unit 14 may store data further indicating a position of the detected key point of the human body in the frame image. Further, the storage unit 14 may store attribute information related to a moving image, for example, a file name of the moving image, a capturing date and time, a capturing place, identification information of a capturing camera, and the like.

Returning to FIG. 4, the screen generation unit 11 generates a UI screen including a playback region playing back and displaying a moving image including a plurality of frame images, and a missing key point display region indicating a key point of a human body not being detected in the human body included in the frame image displayed in the playback region, and causes the display unit 13 to display the generated UI screen.

FIG. 2 illustrates one example of a UI screen. The illustrated UI screen includes a playback region and a missing key point display region. Note that, a manner of layout of the playback region and the missing key point display region is not limited to the illustrated example.

In the playback region, a moving image is played back and displayed. Note that, although not illustrated, buttons performing operations such as playback, pause, rewind, fast forward, slow playback, and stop may be displayed on the UI screen.

In the missing key point display region, information indicating a key point of a human body not being detected in the human body included in the frame image displayed in the playback region is displayed. For example, as in the example illustrated in FIG. 2, a human body model in which a key point being detected and a key point not being detected are identified and displayed may be displayed. An object K₁ outlined by a solid line corresponds to the key point being detected, and an object K₂ outlined by a broken line corresponds to the key point not being detected. A method of identifying and displaying the object K₁ and the object K₂ is not limited to a method in which a mode of an outline is made different, color, a shape, a size, brightness, and the like of an object may be made different, or another method may be adopted. Further, an object as illustrated in FIG. 2 may be displayed corresponding to only one of the key point being detected and the key point not being detected, and an object corresponding to the other key point may be hidden.

Note that, a human body model displayed in the missing key point display region indicates a key point of a human body not being detected, and does not indicate a pose of the human body. Thus, a pose of the human body model displayed in the missing key point display region is always the same pose, and does not change according to a pose of a human body included in the frame image displayed in the playback region. Note that, in the following example embodiments, an example in which a human body model displayed in the missing key point display region indicates a pose of a human body included in the frame image displayed in the playback region will be described.

As another example of information displayed in the missing key point display region, in addition to or instead of a human body model as illustrated in FIG. 2, at least one of “the number of key points not being detected, or the number of key points being detected” and “a name (a head, a neck, or the like) of a key point not being detected, or a name of a key point being detected” may be displayed in the missing key point display region.

Further, in a case where a plurality of human bodies are included in a frame image displayed in the playback region, the screen generation unit 11 may select one human body from the plurality of human bodies in accordance with a predetermined rule, and display a key point of a human body not being detected in the selected human body in the missing key point display region. Examples of the rule for selecting one human body include, but are not limited to, “select a human body specified by a user”, “select a human body having a largest size in a frame image”, and the like. In this case, the screen generation unit 11 may highlight the selected human body on the frame image displayed in the playback region. For example, the screen generation unit 11 may highlight the selected human body by superimposing and displaying a frame surrounding the human body, a mark associated to the human body, or the like on the frame image.

As a modification example, in a case where a plurality of human bodies are included in a frame image displayed in the playback region, the screen generation unit 11 may display a key point of the human body not being detected in each of the plurality of human bodies in the missing key point display region at a time. For example, the screen generation unit 11 may display “a human body model displayed in the missing key point display region in FIG. 2”, “the number of key points not being detected, or the number of key points being detected”, or “a name of a key point not being detected, or a name of a key point being detected” associated to each of the plurality of human bodies included in the frame image displayed in the playback region. In this case, it is preferable to display information indicating a correlation between a plurality of human bodies included in the frame image displayed in the playback region and a detection result of key points of the plurality of human bodies indicated in the missing key point display region. For example, a method such as surrounding “a human body on the playback region” and “a detection result on the missing key point display region” associated to each other with a frame of the same color is conceivable, but the present invention is not limited thereto.

Further, the screen generation unit 11 may always display the information as illustrated in FIG. 2 in the missing key point display region while a moving image is being played back in the playback region. In this case, the information displayed in the missing key point display region is also updated according to switching a frame image displayed in the playback region. In addition, the screen generation unit 11 may display, in the missing key point display region, a key point of a human body not being detected in the human body included in the frame image displayed in the playback region at that time only while a moving image on the playback region is paused.

The screen generation unit 11 can generate the UI screen as described above by using a “result of detection processing of a key point of a human body performed on each of a plurality of frame images included in a moving image” stored in the storage unit 14.

The display unit 13 that displays the UI screen may be a display or a projection apparatus connected to the image processing apparatus 10. In addition, a display or a projection apparatus connected to an external apparatus configured to be communicable with the image processing apparatus 10 may be the display unit 13 that displays the UI screen. In this case, the image processing apparatus 10 serves as a server, and the external apparatus serves as a client terminal. Examples of the external apparatus include, but are not limited to, a personal computer, a smart phone, a smart watch, a tablet terminal, a mobile phone, and the like.

Returning to FIG. 4, the input reception unit 12 receives an input specifying a section to be extracted as a template image from a moving image. The section is a part of a time period in a moving image having a time width. For example, a start position and an end position of the section are indicated by an elapsed time from the beginning of the moving image, or the like.

A means for receiving specification of a section to be extracted is not limited, and any technique can be adopted. In a case of the UI screen illustrated in FIG. 2, due to an operation of pressing a determination button associated to an extraction section start position in a state where a frame image at the start position of a section to be extracted is displayed in the playback region, and an operation of pressing a determination button associated to an extraction section end position in a state where a frame image at the end position of the section to be extracted is displayed in the playback region, an input specifying the section to be extracted is made.

In addition, as a means for receiving specification of a section to be extracted, a means for displaying a slide bar indicating a playback time of a moving image, an elapsed time from the beginning, or the like on the UI screen, and receiving specification of the extraction section start position and the extraction section end position on the slide bar may be adopted. In addition, as a means for receiving specification of a section to be extracted, a means for automatically determining, as the extraction section start position, a position at which a user has started playback, and automatically determining, as the extraction section end position, a position at which the user has finished playback may be adopted. In addition, as a means for receiving specification of a section to be extracted, a means for determining, as the extraction section start position, a position before a reference position (reference frame) in a moving image specified by the slide bar or the like by a user by a predetermined frame, and determining, as the extraction section end position, a position after the reference position by a predetermined frame may be adopted.

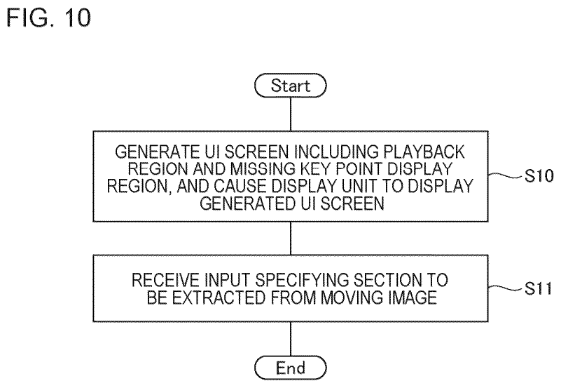

Next, one example of a flow of processing of the image processing apparatus 10 will be described with reference to a flowchart in FIG. 10.

The image processing apparatus 10 generates a UI screen including a playback region playing back and displaying a moving image including a plurality of frame images, and a missing key point display region indicating a key point of a human body not being detected in the human body included in the frame image displayed in the playback region, and causes the display unit 13 to display the generated UI screen (S10). Subsequently, the image processing apparatus 10 receives an input specifying a section to be extracted from the moving image via the UI screen (S11).

Note that, when the image processing apparatus 10 receives an input specifying a section to be extracted from a moving image, the image processing apparatus 10 may cut out the section from the moving image, generate another moving image file, and stored the generated another moving image file. In addition, when the image processing apparatus 10 receives an input specifying a section to be extracted from a moving image, information indicating the specified section may be stored in the storage unit 14. For example, a file name of the moving image, and information indicating the specified section (information indicating the start position and the end position of the section, and the like) may be stored in the storage unit 14 in association with each other.

### “Advantageous Effect”

According to the image processing apparatus 10 of the second example embodiment, for example, as illustrated in FIG. 2, a UI screen including a playback region playing back and displaying a moving image, and a missing key point display region indicating a key point of a human body not being detected in the human body included in a frame image displayed in the playback region can be generated, and the generated UI screen can be displayed on the display unit 13. Then, the image processing apparatus 10 can receive an input specifying a section to be extracted as a template image from the moving image via such a UI screen.

A user can determine a portion in a moving image including a human body having a desired pose or a desired movement and having a good detection state of a key point while referring to the UI screen, and extract the determined portion as a template image. According to the image processing apparatus 10, it is possible to solve a problem of workability of work for preparing a template image having certain quality.

Further, as illustrated in FIG. 2, the image processing apparatus 10 can display a UI screen displaying, in a missing key point display region, a human body model in which a key point being detected and a key point not being detected are identified and displayed. Through such a human body model, a user can intuitively and easily recognize a key point not being detected.

### Third Example Embodiment

An image processing apparatus 10 according to a third example embodiment is different from the image processing apparatus 10 according to the first and second example embodiments in a point that a UI screen further displaying a human body model indicating a pose of a human body included in a frame image displayed in a playback region is generated and displayed, in addition to information (a playback region, a missing key point display region) described in the first and second example embodiments. Hereinafter, it is described in detail.

In addition to information (a playback region, a missing key point display region) described in the first and second example embodiments, a screen generation unit 11 generates a UI screen further displaying a human body model indicating a pose of a human body included in a frame image displayed in the playback region, and causes a display unit 13 to display the generated UI screen. The UI screen displays that a human body model 300 illustrated in FIG. 5 makes a predetermined pose as illustrated in FIGS. 6 to 8. The screen generation unit 11 executes at least one piece of first to third processing described below.

### “First Processing”

In the first processing, the screen generation unit 11 generates a UI screen further including a human body model display region separately from the playback region and the missing key point display region. In the human body model display region, a human body model that is configured by a key point detected in a human body included in a frame image displayed in the playback region and indicates a pose of the human body is displayed.

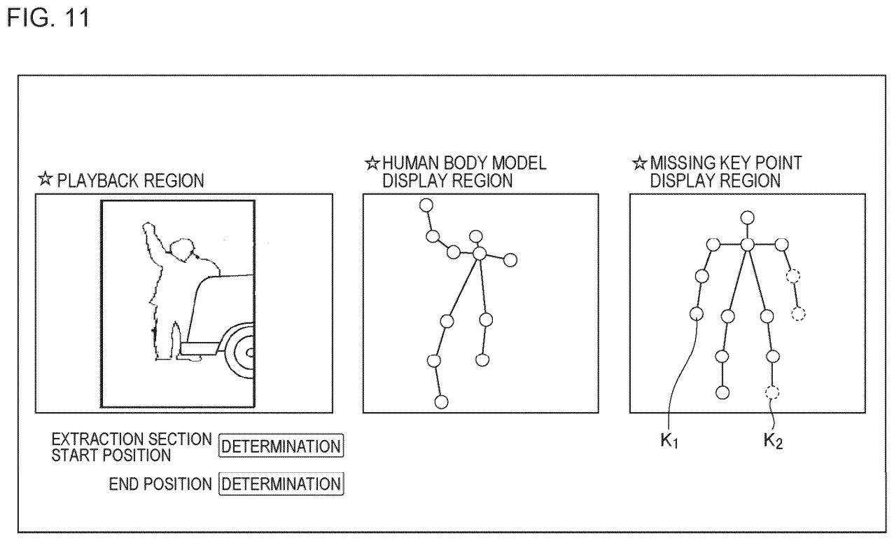

FIG. 11 illustrates one example of the UI screen. Although a human body model is displayed in both the human body model display region and the missing key point display region, it is different from each other in a point that a human body model displayed in the human body model display region indicates a pose of a human body, and a human body model displayed in the missing key point display region indicates a key point not being detected.

Note that, in a case where a plurality of human bodies are included in a frame image displayed in the playback region, the screen generation unit 11 may select one human body from the plurality of human bodies in accordance with a predetermined rule, and display a human body model indicating a pose of the selected human body in the human body model display region. Examples of the rule for selecting one human body include, but are not limited to, “select a human body specified by a user”, “select a human body having a largest size in a frame image”, and the like. In this case, the screen generation unit 11 may highlight the selected human body on the frame image displayed in the playback region. For example, the screen generation unit 11 may highlight the selected human body by superimposing and displaying a frame surrounding the human body, a mark associated to the human body, or the like on the frame image.

As a modification example, in a case where a plurality of human bodies are included in a frame image displayed in the playback region, the screen generation unit 11 may display a plurality of human body models indicating a pose of each of the plurality of human bodies in the human body model display region. In this case, it is preferable to display information indicating a correlation between a plurality of human bodies included in the frame image displayed in the playback region and a plurality of human body models displayed in the human body model display region. For example, a method such as surrounding “a human body on the playback region” and “a human body model on the human body model display region” associated to each other with a frame of the same color is conceivable, but the present invention is not limited thereto.

Further, the screen generation unit 11 may always display a human body model in the human body model display region while a moving image is being played back in the playback region. In this case, a pose of the human body model displayed in the human body model display region is also updated according to switching a frame image displayed in the playback region. In addition, the screen generation unit 11 may display, in the human body model display region, a human body model indicating a pose of the human body included in the frame image displayed in the playback region at that time only while a moving image on the playback region is paused.

### “Second Processing”

In the second processing, the screen generation unit 11 generates a UI screen in which a human body model indicating a pose of a human body is superimposed and displayed on a frame image displayed in the playback region. The human body model may be superimposed and displayed on the human body included in the frame image.

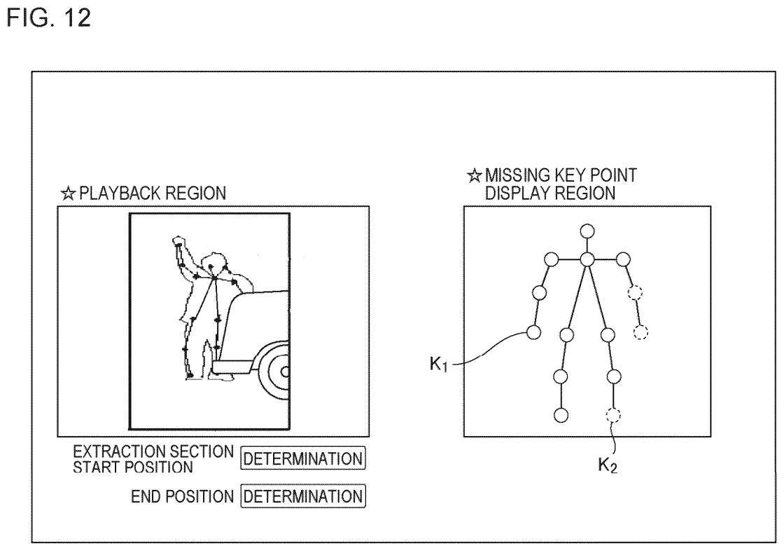

FIG. 12 illustrates one example of the UI screen. A human body model indicating a pose of a human body included in a frame image is superimposed and displayed on the frame image displayed in the playback region. The human body model is superimposed and displayed on the human body included in the frame image.

Note that, in a case where a plurality of human bodies are included in a frame image displayed in the playback region, the screen generation unit 11 may superimpose and display a plurality of human body models indicating a pose of each of the plurality of human bodies on the frame image. Each of the plurality of human body models is preferably superimposed and displayed on the associated human body.

Further, the screen generation unit 11 may always display a human body model on the frame image while a moving image is being played back in the playback region. In this case, a pose and a position of the human body model superimposed and displayed on the frame image are also updated according to switching a frame image displayed in the playback region. In addition, the screen generation unit 11 may superimpose and display, on the frame image, a human body model indicating a pose of the human body included in the frame image displayed in the playback region at that time only while a moving image on the playback region is paused.

### “Third Processing”

In the third processing, the screen generation unit 11 displays, in the missing key point display region, a human body model indicating a pose of a human body, while indicating a key point of the human body not being detected. In this case, a pose of the human body model displayed in the missing key point display region changes according to a pose of a human body included in a frame image displayed in the playback region. Specifically, the pose of the human body model displayed in the missing key point display region becomes the same pose as the pose of the human body included in the frame image displayed in the playback region.

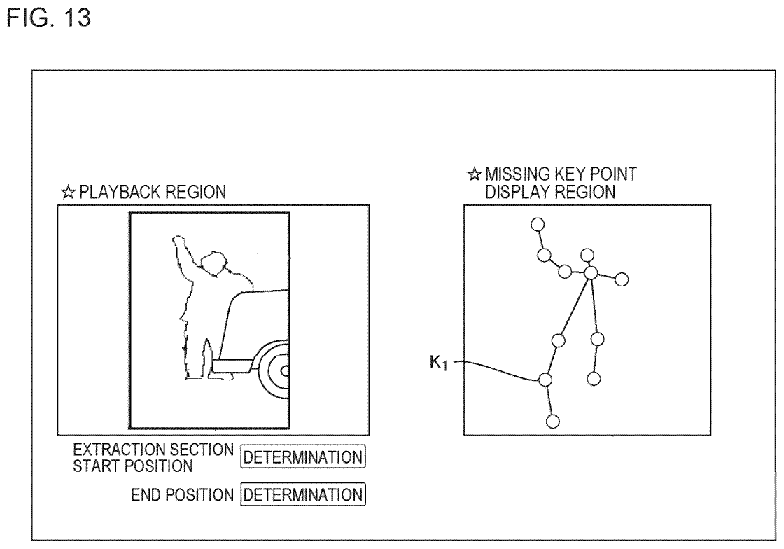

FIG. 13 illustrates one example of the UI screen. A pose of a human body model displayed in the missing key point display region becomes the same pose as a pose of a human body included in a frame image displayed in the playback region.

Note that, in a case where a plurality of human bodies are included in a frame image displayed in the playback region, the screen generation unit 11 may select one human body from the plurality of human bodies in accordance with a predetermined rule, and display a detection result of a key point of the selected human body and a human body model indicating a pose in the missing key point display region. Examples of the rule for selecting one human body include, but are not limited to, “select a human body specified by a user”, “select a human body having a largest size in a frame image”, and the like. In this case, the screen generation unit 11 may highlight the selected human body on the frame image displayed in the playback region. For example, the screen generation unit 11 may highlight the selected human body by superimposing and displaying a frame surrounding the human body, a mark associated to the human body, or the like on the frame image.

As a modification example, in a case where a plurality of human bodies are included in a frame image displayed in the playback region, the screen generation unit 11 may display a detection result of a key point of each of the plurality of human bodies and a plurality of human body models indicating a pose in the missing key point display region. In this case, it is preferable to display information indicating a correlation between a plurality of human bodies included in the frame image displayed in the playback region and a plurality of human body models displayed in the missing key point display region. For example, a method such as surrounding “a human body on the playback region” and “a human body model on the missing key point display region” associated to each other with a frame of the same color is conceivable, but the present invention is not limited thereto.

Further, the screen generation unit 11 may always display a human body model in the missing key point display region while a moving image is being played back in the playback region. In this case, a content (a pose or a detection result of a key point) of the human body model displayed in the missing key point is also updated according to switching a frame image displayed in the playback region. In addition, the screen generation unit 11 may display, in the missing key point display region, a human body model indicating a pose of a human body or a detection result of a key point included in the frame image displayed in the playback region at that time only while a moving image on the playback region is paused.

Other configurations of the image processing apparatus 10 according to the third example embodiment are similar to those of the image processing apparatus 10 according to the first and second example embodiments.

According to the image processing apparatus 10 of the third example embodiment, an advantageous effect similar to that of the image processing apparatus 10 according to the first and second example embodiments is achieved. Further, according to the image processing apparatus 10 of the third example embodiment, it is possible to generate a UI screen further displaying a human body model indicating a pose of a human body included in a frame image displayed in the playback region, and display the generated UI screen.

A user can determine a portion in a moving image including a human body having a desired pose or a desired movement, having a good detection state of a key point, and indicating a correct pose or movement by a detected key point (i.e., detecting a correct key point) while referring to the UI screen, and extract the determined portion as a template image. According to the image processing apparatus 10, it is possible to solve a problem of workability of work for preparing a template image having certain quality.

### Fourth Example Embodiment

An image processing apparatus 10 according to a fourth example embodiment is different from the image processing apparatus 10 according to the first to third example embodiments in a point that a UI screen further displaying a floor map indicating an installation position of a camera in which a moving image is captured is generated and displayed, in addition to information (a playback region, a missing key point display region) described in the first and second example embodiments. The UI screen generated by the image processing apparatus 10 according to the fourth example embodiment may further display information (a human body model indicating a pose of a human body included in a frame image displayed in the playback region) described in the third example embodiment. Hereinafter, it is described in detail.

A screen generation unit 11 generates a UI screen further displaying a floor map indicating an installation position of a camera in which a moving image is captured, in addition to the information (a playback region, a missing key point display region) described in the first and second example embodiments, and causes a display unit 13 to display the generated UI screen. In addition to the above-described information, the screen generation unit 11 may generate a UI screen further displaying the information (a human body model indicating a pose of a human body included in a frame image displayed in the playback region) described in the third example embodiment, and cause the display unit 13 to display the generated UI screen. Hereinafter, some examples of the UI screen including the floor map will be described.

### First Example

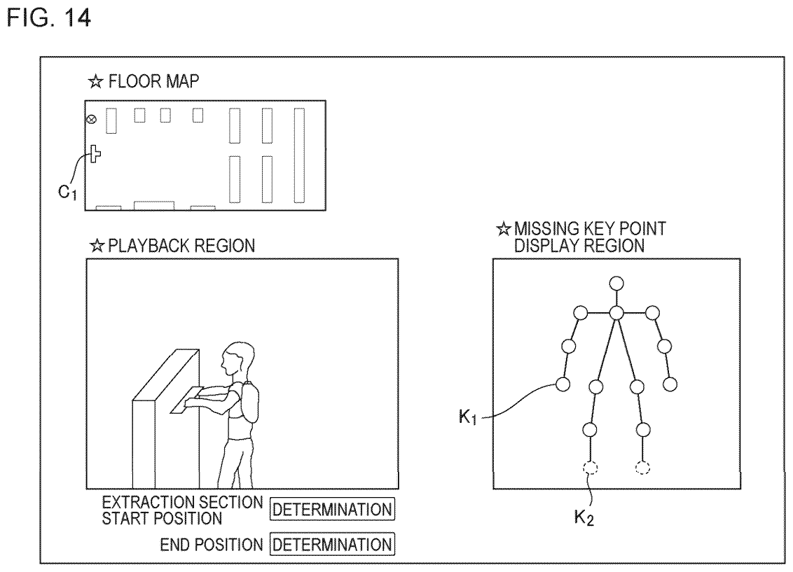

FIG. 14 illustrates one example of a UI screen generated by the screen generation unit 11. In the UI screen illustrated in FIG. 14, a floor map is displayed in addition to the playback region and the missing key point display region. In this example, a camera is installed in a bus. Thus, the floor map is a map in the bus. In the drawing, an icon C₁ indicates an installation position of the camera.

### Second Example

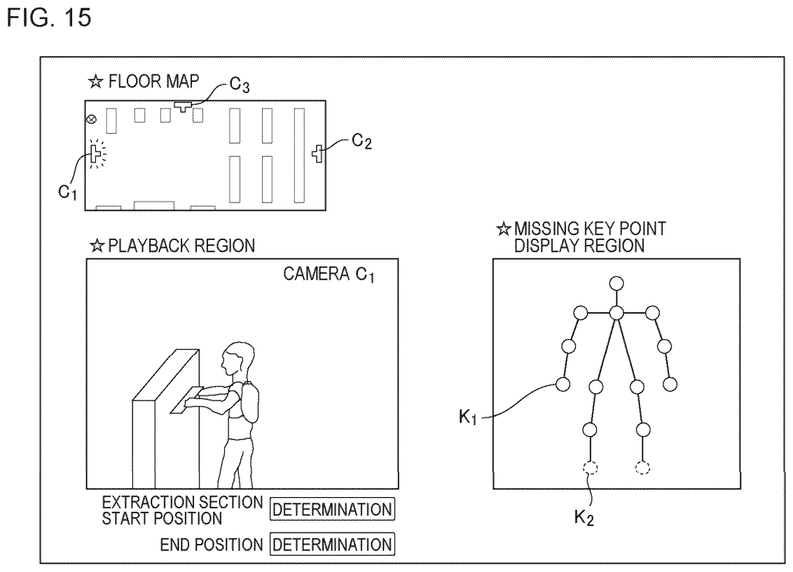

There is a case where a plurality of cameras capture the same place. The plurality of cameras are installed at different places from each other. In this case, as in an example in FIG. 15, the screen generation unit 11 can generate a UI screen including a floor map indicating installation positions of a plurality of cameras. In this example, three cameras are installed in a bus. Then, in the floor map, icons C₁ to C₃ each indicating the installation position of each of the three cameras are illustrated.

In a case of this example, an input reception unit 12 can receive an input specifying one camera. Then, the screen generation unit 11 can play back and display a moving image captured by the camera specified among the plurality of cameras in the playback region. Note that, as illustrated in FIG. 15, the screen generation unit 11 may highlight the specified camera in the floor map. Further, the screen generation unit 11 may display information indicating the specified camera in the playback region. In the example illustrated in FIG. 15, text information identifying the specified camera being a “camera C₁” is superimposed and displayed on the moving image.

A means for receiving an input specifying one camera by the input reception unit 12 varies. For example, the input reception unit 12 may receive an input selecting an icon of one camera on the floor map, or may be achieved by another means.

Note that, the input reception unit 12 may receive an input changing a camera to be specified while a moving image is being played back in the playback region. In this case, in response to an input changing a camera to be specified, a moving image played back and displayed in the playback region is switched from a moving image captured by a camera specified before the change to a moving image captured by a camera specified after the change. At this time, a playback start position of the moving image captured by the camera specified after the change may be determined in response to a playback end position of the moving image that has been played back and displayed before the change. For example, a time stamp indicating a capturing date and time may be added to a moving image captured by a plurality of cameras. Then, in a case where a moving image to be played back and displayed in the playback region is switched in response to the input of changing the camera to be specified during playback of the moving image in the playback region, the input reception unit 12 may first determine the capturing date and time of the playback end position of the moving image that has been played back before the change. Then, the input reception unit 12 may play back the moving image captured by the camera specified after the change from a portion captured at the determined capturing date and time.

### Third Example

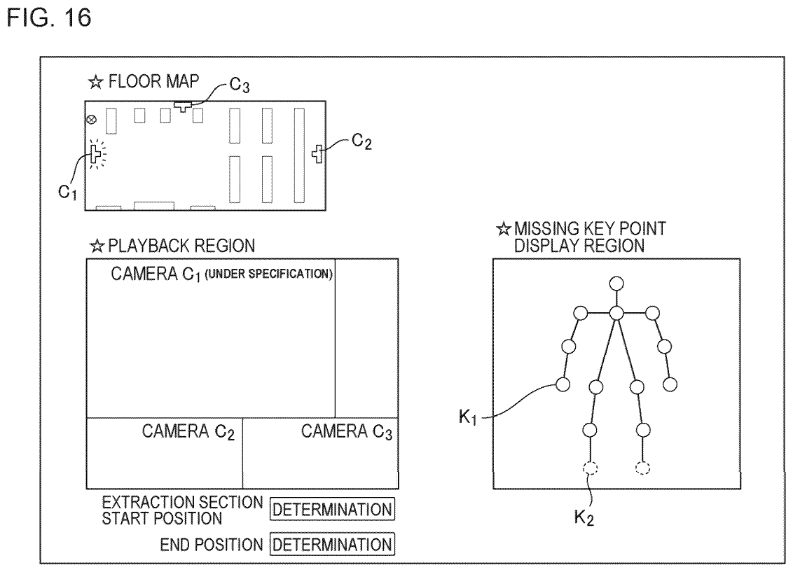

There is a case where a plurality of cameras capture the same place. The plurality of cameras are installed at different places from each other. In this case, as in an example in FIG. 16, the screen generation unit 11 can generate a UI screen including a floor map indicating installation positions of a plurality of cameras. In this example, three cameras are installed in a bus. Then, in the floor map, icons C₁ to C₃ each indicating the installation position of each of the three cameras are illustrated.

In a case of this example, the input reception unit 12 can receive an input specifying one camera. Then, as illustrated in FIG. 16, the screen generation unit 11 can simultaneously play back and display a plurality of moving images captured by each of the plurality of cameras in the playback region, also generate a UI screen highlighting a moving image captured by the specified camera, and cause the display unit 13 to display the generated UI screen. In the illustrated example, the moving image captured by the specified camera is displayed on a larger screen than the moving image captured by the other cameras, and is highlighted by superimposing and displaying text information “under specification” on the moving image, but highlighting may be achieved by using another method.

Further, a time stamp indicating the capturing date and time may be added to the moving images captured by the plurality of cameras. Then, the screen generation unit 11 may synchronize, by using the time stamp, playback timing and the playback positions of a plurality of moving images in such a way that frame images captured at same timing are simultaneously displayed in the playback region.

Note that, as illustrated in FIG. 16, the screen generation unit 11 may highlight the specified camera in the floor map.

A means for receiving an input specifying one camera by the input reception unit 12 varies. For example, the input reception unit 12 may receive an input selecting an icon of one camera on the floor map, may receive an input selecting a moving image captured by one camera on the playback region, or may be achieved by another means.

Note that, the input reception unit 12 may receive an input changing a camera to be specified while a moving image is being played back in the playback region. In this case, in response to an input changing a camera to be specified, the moving image highlighted in the playback region is switched.

In a case of the third example, in the missing key point display region, information on a key point of a human body detected in a moving image captured by the specified camera among a plurality of moving images played back and displayed in the playback region may be displayed. Further, in a case where a configuration according to the third example embodiment is adopted, a human body model indicating a pose of a human body detected in a moving image captured by the specified camera among a plurality of moving images played back and displayed in the playback region may be displayed on the UI screen.

Further, in the case of the third example, when the input reception unit 12 receives a user input specifying one human body on one moving image displayed in the playback region, the screen generation unit 11 may highlight (surround with a frame, or the like) the human body capturing in another moving image. Determination of the same person being captured across a plurality of moving images is achieved by face collation, appearance collation, position collation, or the like.

### Fourth Example

The screen generation unit 11 may further indicate, on a floor map of the first to third examples, a position of a human body detected in a frame image displayed in the playback region. Further, the screen generation unit 11 may further indicate, on the floor map of the first to third examples, a position of a human body detected in a frame image captured by another camera at same timing as the frame image displayed in the playback region.

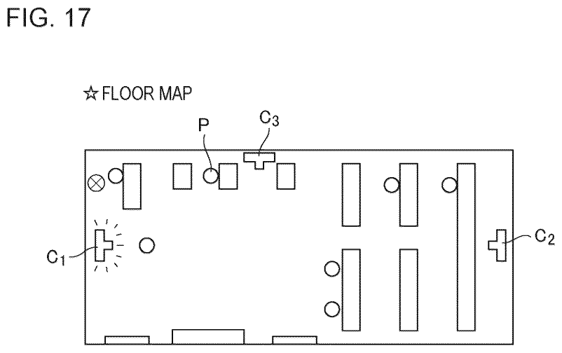

FIG. 17 illustrates one example of a floor map displayed on a UI screen. An icon P indicates a position of a human body. The position of the human body can be determined by an image analysis. For example, in a case where an installation position and an orientation of a camera are fixed, correlation information indicating a correlation between a position in the frame image captured by each of the plurality of cameras and a position in the floor map can be generated in advance. Then, a position of a human body detected in the frame image can be converted into a position on the floor map by using the correlation information.

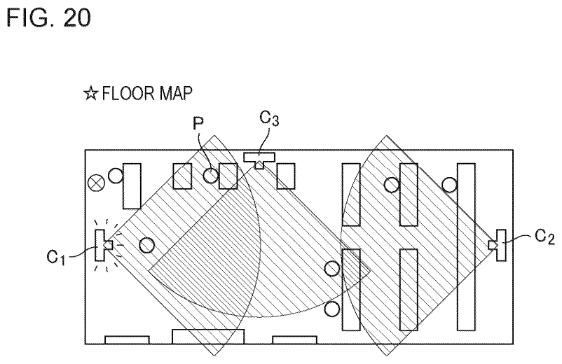

Further, as illustrated in FIG. 20, information indicating a measure of a capturing range of each camera may be displayed on the floor map. In an example illustrated in FIG. 20, the capturing range of each camera is illustrated by a sector figure, but the present invention is not limited thereto. Further, in the example illustrated in FIG. 20, the capturing ranges of all the cameras are displayed, but only the capturing range of the specified camera may be displayed. The capturing range of each camera may be automatically determined from the specifications (an installation position, an orientation, a specification (angle of view, and the like), and the like) of each camera, or may be manually defined. Whether to include, in the capturing range, a position where it is difficult to detect a skeleton because a person is captured by the camera but is captured in small due to a distance, or a position where an obstacle interferes is free, and depending on definition of the capturing range.

Note that, although an example in which an inside of a bus is captured has been described herein, a capturing place is not limited to this example.

Other configurations of the image processing apparatus 10 according to the fourth example embodiment are similar to those of the image processing apparatus 10 according to the first to third example embodiments.

According to the image processing apparatus 10 of the fourth example embodiment, an advantageous effect similar to that of the image processing apparatus 10 according to the first to third example embodiments is achieved. Further, according to the image processing apparatus 10 of the fourth example embodiment, a user can determine a portion to be extracted as a template image while confirming a position of a camera used for capturing, confirming moving images captured by the camera at the same time while switching the moving images, comparing moving images captured by the camera at the same time, or confirming a positional relationship between a human body and the camera. According to the image processing apparatus 10, it is possible to solve a problem of workability of work for preparing a template image having certain quality.

### Fifth Example Embodiment

In a fifth example embodiment, a camera is installed inside a moving object. Then, an image processing apparatus 10 according to the fifth example embodiment is different from the image processing apparatus 10 according to the first to fourth example embodiments in a point that a UI screen further including a moving object state display region indicating a state of a moving object at timing when a frame image displayed in a playback region is captured is generated and displayed, in addition to information (a playback region, a missing key point display region) described in the first and second example embodiments. The UI screen generated by the image processing apparatus 10 according to the fifth example embodiment may further display at least one of information (a human body model indicating a pose of a human body included in a frame image displayed in the playback region) described in the third example embodiment, and information (a floor map) described in the fourth example embodiment. Hereinafter, it is described in detail.

A screen generation unit 11 generates a UI screen further including a moving object state display region, in addition to the information (a playback region, a missing key point display region) described in the first and second example embodiments, and causes a display unit 13 to display the generated UI screen. In addition to the above-described information, the screen generation unit 11 may further generate a UI screen further displaying at least one of the information (a human body model indicating a pose of a human body included in a frame image displayed in the playback region) described in the third example embodiment, and the information (a floor map) described in the fourth example embodiment, and cause the display unit 13 to display the generated UI screen.

In the fifth example embodiment, a camera is installed inside a moving object. The moving object is an object on which a person can ride, and examples thereof include, for example, a bus, a train, an airplane, a ship, a vehicle, and the like. In the moving object state display region, information indicating a state of the moving object at timing when a frame image displayed in the playback region is captured is displayed.

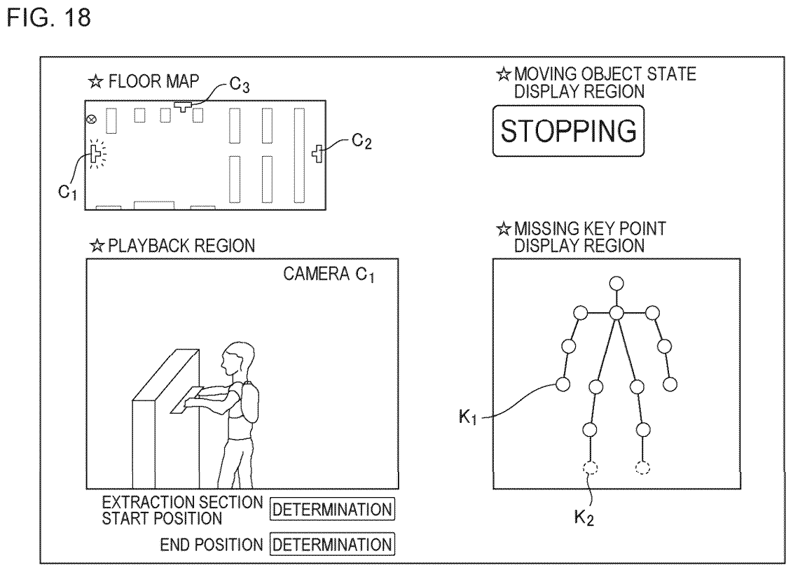

FIG. 18 illustrates one example of a UI screen generated by the screen generation unit 11. On the UI screen illustrated in FIG. 18, a moving object state display region is displayed. Then, in this region, text information being “stopping” is displayed as the state of the moving object at timing when a frame image displayed in the playback region is captured.

The state of the moving object is a state that can be determined by a sensor installed in the moving object. Various states can be defined as a state being displayed in the moving object state display region. For example, examples include, but are not limited to, stopping, under suspension, traveling, moving, traveling straight ahead at less than X₁ km/h, traveling straight ahead at equal to or more than X₁ km/h, turning right, turning left, rotating right, rotating left, raising, lowering, and the like.

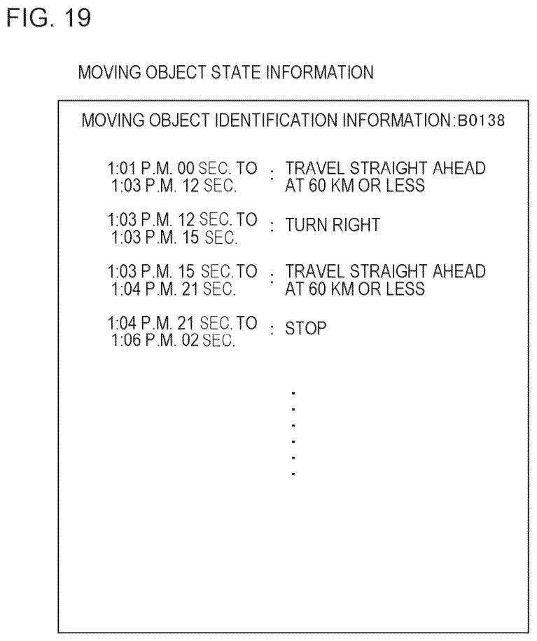

Based on information acquired by various sensors installed in the moving object, the moving object state information indicating the state of the moving object at each piece of timing as illustrated in FIG. 19 can be generated, and stored in a storage unit 14. Based on the moving object state information, the screen generation unit 11 can determine the state of the moving object at timing when the frame image displayed in the playback region is captured, and display information indicating the determined state in the moving object state display region.

Other configurations of the image processing apparatus 10 according to the fifth example embodiment are similar to those of the image processing apparatuses 10 according to the first to fourth example embodiments.

According to the image processing apparatus 10 of the fifth example embodiment, an advantageous effect similar to that of the image processing apparatus 10 according to the first to fourth example embodiments is achieved. Further, according to the image processing apparatus 10 of the fifth example embodiment, a user can determine a portion to be extracted as a template image while confirming a state of a moving object at captured timing. According to the image processing apparatus 10, it is possible to solve a problem of workability of work for preparing a template image having certain quality.

### MODIFICATION EXAMPLE

### First Modification Example

In the above-described example embodiment, image analysis processing such as processing of detecting a key point in advance for a moving image is performed, a result thereof is stored in a storage unit 14, and a characteristic UI screen is generated by using the stored data. As a modification example, when a moving image is played back and displayed in a playback region, image analysis processing such as processing of detecting a key point for the moving image may be performed at that timing, and a UI screen may be generated by using the result.

### Second Modification Example

By using an image analysis technique such as person tracking, the same person being captured across a plurality of frame images in a moving image may be determined. Then, when a user specified one human body capturing in a certain frame image, a screen generation unit 11 may determine another frame image capturing a human body of the same person as the specified human body, whose detection result of a key point is better than that of the specified human body, and display the determined frame image as another candidate on the UI screen.

In addition, the screen generation unit 11 may determine another frame image capturing a human body of the same person as the specified human body, whose detection result of a key point is better than that of the specified human body, and whose pose is the same as a pose of the specified human body or a degree of similarity is equal to or more than a threshold value, and display the determined frame image as another candidate on the UI screen.

Note that, a frame image before a predetermined frame and a frame image after a predetermined frame of the frame image in which the specified human body is captured may be narrowed down as a target for searching for the another candidate.

A “human body having a better detection result of a key point than that of a specified human body” is a human body or the like having a larger number of detected key points than that of the specified human body. The degree of similarity of a pose can be computed by using a method disclosed in Patent Document 1.

“Specification of one human body capturing in a certain frame image” may be achieved, for example, by an operation of specifying one of human bodies capturing in a frame image displayed in the playback region at that time in a state where a moving image displayed in the playback region is paused.

Although the example embodiments of the present invention have been described above with reference to the drawings, these are examples of the present invention, and various configurations other than the above may be adopted.

Further, in the plurality of flowcharts used in the above description, a plurality of steps (pieces of processing) are described in order, but the execution order of the steps executed in each example embodiment is not limited to the order described. In each of the example embodiments, the order of the steps illustrated can be changed within a range that does not interfere with the contents. Further, the above-described example embodiments can be combined within a range in which the contents do not conflict with each other.

Some or all of the above-described example embodiments may be described as the following supplementary notes, but are not limited thereto.
1. An image processing apparatus including:
   a screen generation unit that generates a screen including a playback region playing back and displaying a moving image including a plurality of frame images, and a missing key point display region indicating a key point of a human body not being detected in a human body included in the frame image displayed in the playback region, and causes a display unit to display the generated screen; and an input reception unit that receives an input specifying a section to be extracted from the moving image.
2. The image processing apparatus according to supplementary note 1, wherein
   the screen generation unit generates the screen further displaying a human body model indicating a pose of a human body included in the frame image displayed in the playback region.
3. The image processing apparatus according to supplementary note 2, wherein
   the screen generation unit generates the screen further including a human body model display region displaying a human body model that is configured by the key point detected in a human body included in the frame image displayed in the playback region and indicates a pose of the human body.
4. The image processing apparatus according to supplementary note 2, wherein
   the screen generation unit generates the screen in which a human body model that is configured by the key point detected in a human body included in the frame image displayed in the playback region and indicates a pose of the human body is superimposed and displayed on the frame image displayed in the playback region.
5. The image processing apparatus according to supplementary note 2, wherein
   the screen generation unit generates the screen in which the key point being detected in a human body included in the frame image displayed in the playback region and the key point not being detected are identified and displayed in the missing key point display region, and a human body model indicating a pose of the human body is displayed.
6. The image processing apparatus according to any one of supplementary notes 1 to 5, wherein
   the screen generation unit generates the screen including a floor map indicating an installation position of a plurality of cameras,
   the input reception unit receives an input specifying one of the cameras, and
   the screen generation unit plays back and displays the moving image captured by the specified camera in the playback region.
7. The image processing apparatus according to supplementary note 6, wherein
   the screen generation unit generates the screen highlighting the specified camera on the floor map.
8. The image processing apparatus according to any one of supplementary notes 1 to 5, wherein
   the screen generation unit generates the screen in which a floor map indicating an installation position of a plurality of cameras is further included, and a plurality of the moving images captured by each of a plurality of the cameras are simultaneously played back and displayed in the playback region,
   the input reception unit receives an input specifying one of the moving images in the playback region, and
   the screen generation unit generates the screen highlighting, on the floor map, the camera capturing the specified moving image.
9. The image processing apparatus according to any one of supplementary notes 6 to 8, wherein
   the floor map further indicates a position of a human body detected in the frame image displayed in the playback region.
10. The image processing apparatus according to supplementary note 9, wherein
   the floor map further indicates a position of a human body detected in the frame image captured by another of the cameras at same timing as the frame image displayed in the playback region.
11. The image processing apparatus according to any one of supplementary notes 1 to 10, wherein
   the moving image indicates a scene of an inside of a moving object, and
   the screen generation unit generates the screen further including a moving object state display region indicating a state of the moving object at timing when the frame image displayed in the playback region is captured.
12. An image processing method including,
   by a computer:
      generating a screen including a playback region playing back and displaying a moving image including a plurality of frame images, and a missing key point display region indicating a key point of a human body not being detected in a human body included in the frame image displayed in the playback region, and causing a display unit to display the generated screen; and
      receiving an input specifying a section to be extracted from the moving image.
13. A storage medium storing a program causing a computer to function as:
   a screen generation unit that generates a screen including a playback region playing back and displaying a moving image including a plurality of frame images, and a missing key point display region indicating a key point of a human body not being detected in a human body included in the frame image displayed in the playback region, and causes a display unit to display the generated screen; and
   an input reception unit that receives an input specifying a section to be extracted from the moving image.

### REFERENCE SIGNS LIST

10 Image processing apparatus
11 Screen generation unit
12 Input reception unit
13 Display unit
14 Storage unit
1A Processor
2A Memory
3A Input/Output I/F
4A Peripheral circuit
5A Bus

What is claimed is:

1. An image processing apparatus comprising:
at least one memory configured to store one or more instructions; and
at least one processor configured to execute the one or more instructions to:
generate a screen including a playback region playing back and displaying a moving image including a plurality of frame images, and a missing key point display region indicating a key point of a human body not being detected in a human body included in the frame image displayed in the playback region, and cause a display unit to display the generated screen; and
receive an input specifying a section to be extracted from the moving image.

2. The image processing apparatus according to claim 1, wherein
the at least one processor is further configured to execute the one or more instructions to generate the screen further displaying a human body model indicating a pose of a human body included in the frame image displayed in the playback region.

3. The image processing apparatus according to claim 2, wherein
the at least one processor is further configured to execute the one or more instructions to generate the screen further including a human body model display region displaying a human body model that is configured by the key point detected in a human body included in the frame image displayed in the playback region and indicates a pose of the human body.

4. The image processing apparatus according to claim 2, wherein
the at least one processor is further configured to execute the one or more instructions to generate the screen in which a human body model that is configured by the key point detected in a human body included in the frame image displayed in the playback region and indicates a pose of the human body is superimposed and displayed on the frame image displayed in the playback region.

5. The image processing apparatus according to claim 2, wherein
the at least one processor is further configured to execute the one or more instructions to generate the screen in which the key point being detected in a human body included in the frame image displayed in the playback region and the key point not being detected are identified and displayed in the missing key point display region, and a human body model indicating a pose of the human body is displayed.

6. The image processing apparatus according to claim 1, wherein the at least one processor is further configured to execute the one or more instructions to
generate the screen including a floor map indicating an installation position of a plurality of cameras,
receive an input specifying one of the cameras, and
play back and display the moving image captured by the specified camera in the playback region.

7. The image processing apparatus according to claim 6, wherein
the at least one processor is further configured to execute the one or more instructions to generate the screen highlighting the specified camera on the floor map.

8. The image processing apparatus according to claim 1, wherein the at least one processor is further configured to execute the one or more instructions to
generate the screen in which a floor map indicating an installation position of a plurality of cameras is further included, and a plurality of the moving images captured by each of a plurality of the cameras are simultaneously played back and displayed in the playback region,
receive an input specifying one of the moving images in the playback region, and
generate the screen highlighting, on the floor map, the camera capturing the specified moving image.

9. The image processing apparatus according to claim 6, wherein
the floor map further indicates a position of a human body detected in the frame image displayed in the playback region.

10. The image processing apparatus according to claim 9, wherein
the floor map further indicates a position of a human body detected in the frame image captured by another of the cameras at same timing as the frame image displayed in the playback region.

11. The image processing apparatus according to claim 1, wherein
the moving image indicates a scene of an inside of a moving object, and
the at least one processor is further configured to execute the one or more instructions to generate the screen further including a moving object state display region indicating a state of the moving object at timing when the frame image displayed in the playback region is captured.

12. An image processing method comprising,
by a computer:
generating a screen including a playback region playing back and displaying a moving image including a plurality of frame images, and a missing key point display region indicating a key point of a human body not being detected in a human body included in the frame image displayed in the playback region, and causing a display unit to display the generated screen; and
receiving an input specifying a section to be extracted from the moving image.

13. A non-transitory storage medium storing a program causing a computer to:
generate a screen including a playback region playing back and displaying a moving image including a plurality of frame images, and a missing key point display region indicating a key point of a human body not being detected in a human body included in the frame image displayed in the playback region, and cause a display unit to display the generated screen; and
receive an input specifying a section to be extracted from the moving image.

14. The image processing method according to claim 12, wherein
the computer generates the screen further displaying a human body model indicating a pose of a human body included in the frame image displayed in the playback region.

15. The image processing method according to claim 14, wherein
the computer generates the screen further including a human body model display region displaying a human body model that is configured by the key point detected in a human body included in the frame image displayed in the playback region and indicates a pose of the human body.

16. The image processing method according to claim 14, wherein
the computer generates the screen in which a human body model that is configured by the key point detected in a human body included in the frame image displayed in the playback region and indicates a pose of the human body is superimposed and displayed on the frame image displayed in the playback region.

17. The image processing method according to claim 14, wherein
the computer generates the screen in which the key point being detected in a human body included in the frame image displayed in the playback region and the key point not being detected are identified and displayed in the missing key point display region, and a human body model indicating a pose of the human body is displayed.

18. The non-transitory storage medium according to claim 13, wherein
the program causing the computer to generate the screen further displaying a human body model indicating a pose of a human body included in the frame image displayed in the playback region.

19. The non-transitory storage medium according to claim 18, wherein
the program causing the computer to generate the screen further including a human body model display region displaying a human body model that is configured by the key point detected in a human body included in the frame image displayed in the playback region and indicates a pose of the human body.

20. The non-transitory storage medium according to claim 18, wherein
the program causing the computer to generate the screen in which a human body model that is configured by the key point detected in a human body included in the frame image displayed in the playback region and indicates a pose of the human body is superimposed and displayed on the frame image displayed in the playback region.
